SEQUENCE LISTING (7 sequences)

SEQ ID NO: 1 - DNA, 246 bp, Homo sapiens
atgctactagtaaatcagtcacaccaaggcttcaataaggaacacacaagcaagatggtaagcgctattgttttatatgtgcttttggcggcggcggcgcattctgcctttgcggcggatcttggatctttggacaagaggcaccatcaccatcaccatgcgggcgtcgaggaggtagcggcctccgggagccacctgaatggcgacctggatccatctagactcgagattaatgaattccggagc

SEQ ID NO: 2 - protein, 72 aa, Homo sapiens
MLLVNQSHQGFNKEHTSKMVSAIVLYVLLAAAAHSAFAADLGSLDKRHHHHHHAGVEEVAASGSHLNGDLDP

SEQ ID NO: 3 - DNA, 67 bp, Artificial Sequence
  Primer
attaatagatctttggacaagaggcaccatcaccatcaccatgcgggcgtggaggaggtagcggcct

SEQ ID NO: 4 - DNA, 53 bp, Artificial Sequence
  Primer
attaatctcgagtctagacggtccgttaatagtcatctcctctgctgacaact

SEQ ID NO: 5 - DNA, 30 bp, Artificial Sequence
  Primer
cgttaaaattgagacatgaagtgaggccgt

SEQ ID NO: 6 - DNA, 35 bp, Artificial Sequence
  Primer
attaatggatccagcggccgtggagacgcgggtgt

SEQ ID NO: 7 - DNA, 46 bp, Artificial Sequence
  Primer
attaatctcgaggaattcttaaaattgagacatgaagtgaggccgt

(19) United States
(12) Patent Application Publication
(10) Pub. No.: US 2004/0077031 A1
(43) Pub. Date: Apr. 22, 2004

(54) Novel methionine aminopeptidase-2 and uses thereof

(76) Inventors: Jieyi Wang, Lake Bluff, IL (US); George S. Sheppard, Wilmette, IL (US); PingPing Lou, Gurnee, IL (US); Jack Henkin, Highland Park, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/272,855

(22) Filed: Oct. 17, 2002

(51) Int. Cl. C12Q 1/37; A61K 38/48

(52) U.S. Cl. 435/23; 424/94.63

(57) ABSTRACT

The present invention uses the manganese-dependent physiological form of the enzyme methionine aminopeptidase type 2 to assess inhibition by agents that might be used in the treatment of angiogenesis, cancer, malaria and leishmaniasis. This method has the advantage of using the manganese form of the enzyme and therefore, the advantage of identifying potent inhibitors that might not show activity in cellular systems because the wrong metal cofactor is used. Therefore it is a new tool for the development of agents useful in the therapy of cancer and other angiogenesis-related diseases and, several infectious diseases including malaria, leishmaniais and microsporidiosis.

### FIELD OF THE INVENTION

The present invention relates to a novel, methionine aminopeptidase-2 that uses manganese as the necessary stimulating divalent cation. Methionine aminopeptidase-2 inactivation is linked to anti-angiogenic, anti-tumor, anti-parasitic, anti-bacterial and anti-microsporidian effects. The novel manganese form of the enzyme represents a useful in vitro tool, for example in the discovery of inhibitory compounds that can be used in the treatment of cancer, angiogenesis, malaria and leishmaniasis.

### BACKGROUND OF THE INVENTION

Methionine aminopeptidases (MetAPs) are cellular metalloproteases capable of removing the N-terminal initiator methionine residue of nascent proteins. The removal of the N-terminal methionine is a critical step for protein modifications that are important in controlling protein subcellular localization and/or protein degradation. Inhibition of MetAPs therefore affects regulation of cellular signal transduction and cell cycle progression.

MetAP enzymes have a conserved C-terminal catalytic domain with a protease fold, termed the “pita bread” fold, that appears to be highly conserved in all MetAP enzymes and other related enzymes (Bazan et al., Proc. Nat'l Acad. Sci. USA, 91(7): 2473-77, 1993). The C-terminus of the human MetAP2 contains the catalytic domain showing high amino acid identity with MetAP sequences from prokaryotes and yeast, while the N-terminal region has two basic poly-lysine blocks and an acidic aspartic acid block.

There are two cobalt-dependent MetAP families: Type 1 or MetAP 1, presently composed of the prokaryote and yeast sequences (and represented by the E. coli structure), and Type 2 or MetAP2 represented by human MetAP, the yeast open reading frame, and the partial prokaryotic sequence (Arfin et al., Proc. Nat'l. Acad. Sci. 92(17): 7714-18, 1995). The Type 2 enzymes are distinguished from the Type 1 by a helical subdomain of approximately 60 residues in length inserted in the C-terminal domain (Lowther and Mathews, Biochim. Biophys. Acta 1477:157-167, 2000).

MetAP2 was first described as an enzyme that copurified with the eukaryotic initiation factor 2α (eIF2α) from rabbit reticulocytes, with a molecular weight of 67 kDa (Gupta et al., In: Translational Regulation of Gene Expression (Ilan J., Ed) Vol 2, pp 405-431, Plenum Press, NY, 1993). MetAP2 is a metalloprotease known for its dual functions: (a) methionine aminopeptidase activity, which removes initiator methionine from nascent proteins, and (b) protection of eukaryotic initiation factor 2α (eIF-2α) from phosphorylation inactivation. (For this function, the metalloprotease is referred to as p67.) Both functions are mediated by distinct domains of the protein (Datta, Biochimie 82:95-107, 2000). N-terminal truncation of the highly charged domain of MetAP2, which is involved in the protection of eIF2α from phosphorylation inactivation, does not affect the activity of the enzyme in vitro (Gupta et al., supra; Yang et al., Biochemistry 40(35):10645-54, 2001). Similarly, the MetAP2 is able to protect eIF2α from inactivation even when the methionine aminopeptidase activity is covalently inhibited by the specific inhibitor TNP-470 (Griffith et al., Chem. Biol. 4(6) :461-71, 1997).

Human MetAP2 (hMetAP2) was shown to have methionine aminopeptidase activity when the recombinant protein was made and characterized (Li and Chang, Biochem. Biophys. Res. Comm. 227(1):152-9, 1996). Similar to MetAP2 from Pyrococcus furiosus, hMetAP2 showed a catalytic domain containing two cobalt metal ions in the active center (Liu et al., Science 282(5392):1324-7, 1998). Previous studies describing new compounds as potential anti-angiogenic agents have used assays of functional MetAP in the presence of Co²⁺ as the metal cofactor (U.S. Pat. No. 6,207,704, International Publication No. WO 01/36404, International Publication No. WO 01/24796 and International Publication No. WO 01/78723).

A recent study showed that Zn²⁺, under physiological conditions, was a much better cofactor than Co²⁺ for yeast MetAP1, (Walker and Bradshaw, Protein Sci. 7(12):2684-7,1998). Another study showed that the physiologically relevant metal ion for E. coli MetAP1 was probably Fe²⁺, on the basis of a combination of whole cell metal analyses and activity measurements (D'souza and Holz, Biochemistry 38(34): 11079-85, 1999); however, Fe²⁺ provided a MetAP enzyme that was 80. as active as the Co²⁺ substituted form of the enzyme, and Zn²⁺ induced activity levels almost 10-fold lower than those in the presence of Co²⁺ or Fe^{2+.} (D'souza and Holz, supra). A study using recombinant human MetAP2 concluded that MetAP2 was a Co²⁺-dependent metalloprotease (Li and Chang, Proc. Natl. Acad. Sci USA 92:12357-61, 1995).

Manganese is known to be a catalytically required cofactor in certain metalloproteases involved in mammalian nitrogen and oxygen metabolism. In plants, Mn²⁺ is an essential component of the oxygen-evolving complex of photosystem II in green plants (Christianson D W, Prog. Biophys. Mol. Biol. 67(2-3):217-52, 1997). Arginase, manganese catalases, enolase, superoxide dismutase and serine/threonine protein phosphatase-1 are just a few examples of well-studied manganese enzymes. Aminopeptidase P from E. coli has been shown to contain a binuclear Mn²⁺ core (Wilce et al, Proc. Nat'l. Acad. Sci. 95(7):3472-7, 1998). Aminopeptidase P hydrolyzes amino-terminal X-Pro peptide bonds (where X may be any amino acid). Its biological functions, amino acid sequence and metal specificity are remarkably similar to human prolidase, an enzyme involved in proline recycling for collagen biosynthesis. Aminopeptidase P and prolidase structurally belong to a new protease family with the “pita-bread” fold (Bazan et al, Proc. Nat'l. Acad. Sci. 91(7):2473-7, 1994), which include methionine aminopeptidases. The importance of determining the true metal cofactor used by MetAP2 in vivo is emphasized by the involvement of this metalloprotease in several disease states. By knowing the true metal cofactor used by MetAP2 in vivo, new compounds targeting MetAP2 can be discovered using in vitro methods that include the correct metal cofactor. This would lead to a new arsenal of compounds with high effectiveness in vivo, useful as therapeutic agents.

In view of the above, two things are clear: first, the true identity of the optimal metal ion required by the MetAP2 and the MetAP2's native metal ion content under physiological conditions need to be determined. Many compounds with good cellular penetration are not effective in vivo because the wrong metal cofactor was used in determining their effectiveness in vitro. Second, identification of the physiological metal cofactor for human MetAP2 will provide not only a better understanding of the involvement of the enzyme in the cell cycle but, most importantly, a way to identify compounds that inhibit human MetAP2 both in purified systems and under physiological conditions.

### SUMMARY OF THE INVENTION

One embodiment of the present invention encompasses a method for assaying the activity of an aminopeptidase in the presence of the metal cofactor manganese on a substrate comprising methionine, for a time and under conditions sufficient to allow said aminopeptidase to cleave said substrate in order to release the methionine, wherein the cleavage of said methionine generates a measurable signal proportional to the activity of said aminopeptidase. The present invention specifically encompasses a methionine aminopeptidase type 2. The method includes the use of several oligomeric peptides as substrates, specifically trimeric (methionine-alanine-serine) and octameric peptides (MARCKS proteins and others). Additionally, the method of the present invention specifically includes the use of manganese in divalent form as the metal cofactor for said metalloprotease, specifically the methionine aminopeptidase type 2.

One embodiment of the present invention encompasses the detection of free radioactive methionine released upon enzymatic activity of said methionine aminopeptidase type 2 on a substrate comprising radioactive methionine as a measure of the methionine aminopeptidase type 2 activity in the presence of divalent manganese.

Another embodiment of the present invention encompasses a method involving tetrapeptide comprising methionine and its use in the detection of methionine aminopeptidase activity. HPLC is used to separate free methionine released upon enzymatic activity in the presence of manganese and the resulting tripeptide. The resulting tripeptide is measured by UV absorbance and indicates enzymatic activity.

Additionally, the present invention contemplates the detection of color development resulting from free methionine released from a substrate upon activity of said methionine aminopeptidase type 2 in the presence of divalent manganese, wherein said color development results from oxidation of free methionine. Additionally, the method of the present invention includes a methionine-containing substrate selected from the group consisting of methionine-p-nitroanilide (Met-pNA) and L-methionine 7-amido-4-methylcoumarin (Met-AMC). After the enzymatic action of the methionine aminopeptidase on either of said substrates, color development or a fluorescent signal results from the methionine-free p-nitroanilide (pNA) and the methionine-free 7-amido-4-methylcoumarin (AMC), respectively. Said signals are independently a measure of the methionine aminopeptidase activity in the presence of divalent manganese as the metal cofactor.

The present invention also includes a method for assaying the activity of methionine aminopeptidase, comprising the steps of contacting said methionine aminopeptidase with a first substrate comprising methionine, for example a dipeptide, wherein the cleavage and release of said methionine, in the presence of the metal cofactor manganese results in a compound which is then contacted with a second aminopeptidase, a proline aminopeptidase for example. Said second peptidase is capable of generating a measurable signal that proportionally indicates activity of said methionine aminopeptidase on the dipeptide. The method of the present invention comprises Met-Pro-p-nitroanilide as the preferred dipeptide and a proline aminopeptidase as the second peptidase.

A further embodiment of the present invention includes a method for identifying compounds that inhibit function of methionine aminopeptidase. The activity of the methionine aminopeptidase in the presence of manganese is measured by the amount of radioactive methionine released by the enzymatic activity of the methionine aminopeptidase on a polypeptide comprising radioactive methionine. The method comprises measuring the activity of the methionine aminopeptidase using the same approach described directly above in the presence of test compounds that potentially inhibit methionine aminopeptidase. A decrease in the amount of released radioactive free methionine is an indication of the methionine aminopeptidase decreased enzymatic activity and therefore of the inhibitory effect of the test compound on said methionine aminopeptidase. The isotope in the radioactive methionine-comprising substrate is selected from the group consisting of tritium ³[H]), ³⁵[S] and ¹⁴[C].

An additional embodiment of the present invention includes a method for identifying compounds that inhibit function of aminopeptidase comprising the use of a coupled enzyme assay. The method comprises the steps of contacting the aminopeptidase with a polypeptide comprising methionine in the presence of divalent manganese as a metal cofactor for a time and under conditions sufficient for said aminopeptidase to cleave said methionine from said polypeptide. The cleaved methionine is oxidized by amino acid oxidase (AAO) and hydrogen peroxide (H₂O₂) is generated. The formation of H₂O₂is monitored by its utilization as a substrate for horseradish peroxidase (HRP), which then oxidizes either o-dianisidine or Amplex Red generating a color signal or a fluorescent signal, respectively. The measurable signal is a measure of the amount of methionine cleaved and therefore of the aminopeptidase enzymatic activity. The same assay is performed in the presence of a test compound and the signals generated are compared, a decrease in the signal generated in the presence of a test compound indicates that the compound inhibits the aminopeptidase enzymatic activity.

The invention also includes a method for identifying compounds that inhibit function of aminopeptidase comprising a method using alternative substrates. The method comprises the steps of contacting the aminopeptidase in the presence of manganese as the metal cofactor with the alternative substrates L-methionine p-nitroalniline (Met-pNA) or L-methionine 7-amido-4-methylcoumarin (Met-AMC). After the aminopeptidase has cleaved methionine from either of these substrates, the resulting p-nitroaniline (p-NA) or 7-amido-4-methylcoumarin (AMC) generate a color signal or fluorescence, respectively. Comparing the signals generated in the absence and the presence of a test compound, a decrease in the signal generated indicates that the test compound is an inhibitor of the aminopeptidase when manganese is the metal cofactor.

Another embodiment of the present invention includes a method for identifying compounds that inhibit function of aminopeptidase comprising using HPLC to separate substrate peptides and products and subsequent on-line UV detection, for example, of each separated product. The signals generated in the absence and in the presence of a test compound are compared. A decrease in the signal generated in the presence of the compound indicates that the test compound is an inhibitor of the aminopeptidase when manganese is the metal cofactor.

A further embodiment of the present invention comprises a method for determining intracellular methionine aminopeptidase type 2 inhibition by a compound, wherein said compound inhibits aminopeptidase activity in a test cell comprising endogenous manganese as a metal cofactor. The method comprises the steps of contacting a test cell with labeled methionine for a time and under conditions sufficient to allow the test cell to incorporate said radioactive methionine into its produced proteins, isolating said produced proteins, contacting the aminopeptidase with said proteins and allow the aminopeptidase to cleave methionine from these newly produced proteins. The same protocol is performed in the presence of a test compound and the resulting signals are compared. An increase in the amount of free labeled methionine indicates that said test compound is an inhibitor of intracellular methionine aminopeptidase 2. The cell can be selected from the group consisting of an endothelial cell (HMVEC), a tumor cell and a white blood cell.

An additional embodiment of the present invention includes a method for determining anti-angiogenic activity of a compound in vitro, wherein said compound inhibits aminopeptidase activity in an endothelial cell comprising endogenous manganese as a metal cofactor, comprising the steps of contacting an endothelial cell with a compound that inhibits methionine aminopeptidase activity and determining whether said compound inhibits cell proliferation, wherein lack of proliferation indicates said compound has anti-angiogenic activity. Similarly, the present invention includes a method for determining anti-tumor activity of a compound in vitro, wherein said compound inhibits aminopeptidase activity in a tumor cell comprising endogenous manganese as a metal cofactor, comprising the steps of contacting a tumor cell with a compound that inhibits methionine aminopeptidase activity and determining whether said compound inhibits cell proliferation, wherein lack of proliferation indicates said compound has anti-tumor activity.

A further embodiment of the present invention includes a method of inhibiting methionine aminopeptidase activity in a mammal in need of said inhibition, comprising administering to the mammal a therapeutically effective amount of a compound that inhibits methionine aminopeptidase activity.

A further embodiment of the present invention includes a method of treating or preventing angiogenesis in a mammal in need of said treatment or prevention, comprising administering to said mammal a therapeutically effective amount of a compound that inhibits methionine aminopeptidase activity. Similarly, another embodiment comprises a method of treating or preventing tumor growth in a mammal in need of said treatment or prevention comprising administering to said mammal a therapeutically effective amount of a compound that inhibits methionine aminopeptidase activity.

### BRIEF DESCRIPTION OF THE DRAWINGS

FIG. 1. Top panel illustrates cloning strategy of human MetAP1 and MetAP2 by RT-PCR from endothelial cell total RNA using pAcGP67B baculovirus transfer vector (Pharmingen, San Diego, Calif.). Lower panel illustrates recombinant proteins of MetAP1 and MetAP2 analyzed by SDS-PAGE.

FIG. 2. Shows the metal dependence of MetAP1. A. Metal dependence measured using trimeric MAS as substrate. B. Metal dependence using octameric MGAQFSKT as substrate. Co²⁺ and Mn²⁺, showed maximal stimulation of MetAP1 activity.

FIG. 3. Shows the metal dependence of MetAP2. A. Metal dependence measured using MAS as substrate. B. Metal dependence using MGAQFSKT as substrate. Co²⁺ and Mn²⁺ showed maximal stimulation of MetAP2 activity.

FIG. 4. A. Illustrates the metal dependence of MetAP2 measured with ³H-MASK. B. Illustrates the metal dependence of MetAP1 measured with ³H-MASK. There is a maximal stimulation of MetAP2 activity in the presence of Co²⁺ and Mn²⁺.

FIG. 5. Represents the MetAP2 activity in the presence of selective MetAP2 inhibitor A310840 (application Ser. No. 09/377,261). A310840 did not inhibit the enzyme in the presence of Mn²⁺, and did not inhibit MetAP2 activity inside cells, indicating that the MetAP2 was using Mn²⁺ as a cofactor rather than any other metal.

FIG. 6. Illustrates the inhibition of intracellular MetAP2 activity by fumagillin and A-357300 (U.S. Pat. No. 6,242,494; application Ser. No. 09/833,917).

FIG. 7. Panel A shows the inhibition of angiogenesis by A-357300 in vitro using HMVEC grown in microcarriers. Panels B, C and D show inhibition of angiogenesis by A-357300 in vivo using a mouse cornea neo-vascularization model.

FIG. 8. Shows the inhibition by A-357300 in vivo using the growth of human tumor xenograft in mice.

### DETAILED DESCRIPTION OF THE INVENTION

The subject invention relates to a novel, isolated, physiological form of the human methionine aminopeptidase type 2, referred to herein as hMetAP2, that uses manganese (Mn²⁺ hereinafter) as its metal cofactor. Furthermore, the invention provides a method for identifying agents that inhibit hMetAP2 under conditions that are predictive of the activity of the enzyme in cellular systems. For example, previous studies directed to the identification of inhibitors of the enzyme have used cobalt (Co²⁺ hereinafter) as the metal cofactor. Under these conditions, inhibitors that were potent against the Co²⁺ form of the enzyme might have had very low activity on the physiological form, i.e., “true form”, of hMetAP2 that uses Mn²⁺. The “true form” of the enzyme of the present invention is therefore scientifically more appropriate and accurate when used in the identification of compounds that may become potent anti-cancer, anti-angiogenic, anti-microbial and anti-parasitic therapeutic agents.

### 1. Definitions and Related Information

By “anti-angiogenic compounds” is meant compounds that can inhibit angiogenesis. “Angiogenesis” or “neovascularization” is defined as the formation of new blood vessels into a tissue or an organ. Under normal conditions humans or animals undergo angiogenesis only in very specific and restricted conditions, e.g., wound healing, embryonic development, female reproductive cycle. Angiogenesis, however, can be also abnormal or undesired. Diseases that involve abnormal angiogenesis include, e.g., tumors, diabetic retinopathy, inflammatory diseases and arteriosclerosis.

One of the leading anti-angiogenic compounds, fumagallin (or its derivative TNP-470), inhibits neovascularization by arresting endothelial cell cycle. The mechanism of this inhibition is by covalent binding to MetAP2. Accordingly, MetAP2 is an important target of study in the analysis of potential antiangiogenic compounds (Sin et al., Proc. Natl. Acad. Sci. USA, 94:6099-6103, 1997; Ingber et al., In: Cancer Therapeutics (B. A. Teicher, ed. P. 283-298. Humana Press, Totowa, N.J., 1997; Castronovo and Belotti, Eur. J. Cancer 32A (14):2520-7, 1996). Selective inhibition of MetAP2 enzyme activity by fumagillin or TNP-470 prevents removal of initiator methionine of its specific cellular protein substrate(s) that are essential for the cell cycle progression of the particular cell types.

Angiogenesis inhibitors specific for methionine aminopeptidase have also been shown to block in vitro growth of P. falciparum and Leishmania donovani. Most importantly, in the case of P. falciparum, the cloroquine-resistant strains were equally susceptible to MetAP2 inhibitors (Zhang et al., J. Biochem Sci. 9(1):34-40, 2002).

Reports showing the lethality of deleting the MetAP gene from E. coli and both MetAP types 1 and 2 genes from yeast indicate that antiangiogenic compounds that specifically inhibit MetAP1 and 2 may be also beneficial as antimicrobial and antifungal agents (Chang et al., J. Bacteriol. 171:4071, 1989; Li and Chang, Proc. Nat'l Acad. Sci USA 92:12357-61, 1995). Also, inhibitors of MetAP2 have been shown to be active both in vivo and in vitro against several microsporidia. In humans, microsporidiosis has been associated most often with hepatitis, myositis, keratoconjuntivitis, sinusitis, kidney and urogenital infection, and other disseminated disease states (Weiss et al., J. Eukaryot. Microbiol. 2001; Suppl.:88S-90S).

By “metal cofactor” is meant the biologically relevant metal contained in the catalytic domain of the active center of the enzyme. At this time it is unclear to what degree the metal used in physiological conditions is intrinsic to each enzyme or depends on the metal level in the environment (Lowther and Mathews, Biochem. Biophys. Acta 1477:157-167, 2000). The metal dependence of the MetAPs has been established by the loss of activity upon treatment with a metal chelating agent like ethylene-diamine-tetra-acetic acid (EDTA); however, experiments to determine the physiological metal of the MetAP are controversial. Activity has been observed in the presence of several metals including Zn²⁺, Co²⁺, Fe²⁺ and Mn²⁺ for MetAP1 and MetAP2 (Lowther and Mathews, supra). However, there are no studies available regarding the native metal content and the in vivo metal-dependence of human MetAP2.

The present invention involves an isolated, novel form of hMetAP2 that has been determined to preferably use manganese as a metal cofactor, discovered through extensive comparison of the effect of the most common divalent cations on recombinant human MetAP1 and MetAP2.

The present invention uses recombinant human MetAP1 and MetAP2 cloned from endothelial cell total RNA. The term “recombinant” refers to an artificial combination of two otherwise separated segments of a sequence, either by the chemical synthesis or by the manipulation of isolated segments of DNA or RNA by genetic engineering techniques. The “gene” is the resulting nucleic acid fragment that expresses a specific protein, including the regulatory sequences preceding (5′ non-coding sequences) and following (3′ non-coding sequences) the coding sequences.

### 2. General Methods

### 2.1. Recombinant Human MetAp1 and MetAP2

The recombinant human MetAp2 and MetAp1 were cloned by RT-PCR from endothelial cell total RNA. Coding sequences were cloned in a baculovirus transfer vector and transfected into host cells by methods known by those skilled in the art (See Sambrook et al., “Molecular Cloning: A Laboratory Manual, Second Ed (1989), Cold Spring Harbor Laboratory Press, Cold Spring Harbor, N.Y.). Once the recombinant MetAP1 and MetAP2 were expressed by the host cells, the enzymes were secreted into the culture medium, purified and tested for catalytic activity.

### 2.2. Activity of MetAPs in vitro

The present invention includes methods for analyzing, in particular, the activity of MetAP1 and MetAP2 using different metals as cofactors for the proteolytic activity. Several methods are available to measure MetAP2 activity, including the colorimetric ninhydrin method (Doi et al., Anal. Biochem 118:173-184, 1981), separation of substrate peptides and products by reverse phase HPLC with on-line UV detection of each separated compound (Larrabee et al., Anal. Biochem 269:194-198, 1999) and, direct and indirect spectrophotometric assays.

The methods used in the present invention monitor the formation of free 1-amino acids (methionine) from peptides using indirect spectrophotometric assays. Substrates used in the present invention comprise, for example, the trimeric peptide MAS (methionine-alanine-serine), octameric peptide substrates comprising MARKS proteins (methionine-glycine-alanine-glutamine-phenylalanine-serine-lysine-threonine), PKC-α (methionine-glycine-asparagine-alanine₄-lysine), Src^{p60} (methionine-glycine-serine₂-lysine-serine-lysine-proline) eNOS (methionine-glycine-asparagine-leusine-lysine-serine-valine-alanine) and GAPDH (methionine-glycine-lysine-valine-lysine-valine-glycine-valine). However, oligopeptides of any length can be used as appropriate substrate (See Example 3.1.b and Table 1). After MetAP cleaves methionine from any of these substrates, the free methionine is subjected to amino acid oxidase treatment followed by peroxidase reaction and o-dianisidine color development (Carter and Miller, J. Bacteriol. 159:453-459, 1984, Yang et al., Biochemistry 40:10645-10654, 2001). Alternatively, any of these substrates can be made radioactive by including isotope labeled methionine. Direct measurement of cleaved radioactive methionine reflects MetAP activity. (Details are presented in Examples 3 and 4).

Direct spectrophotometric assays method use the alternative substrates, L-methionine p-nitroaniline (Met-pNA) and L-methionine 7-amido-4-methylcoumarin (Met-AMC). MetAP cleaves methionine from these substrates and the resulting methionine-free substrates produce a measurable signal. The resulting chromophore p-nitroaniline (pNA) is monitored by increased color development (absorbance), and the resulting 7-amido-4-methylcoumarin (AMC) is monitored directly by the increased fluorescence in the samples. Other methods involve the use of Met-Pro-p-nitroanilide substrate; MetAP catalizes the cleavage of methionine and the resulting prolyl-p-nitroanilide is subsequently cleaved by a prolyl-aminopeptidase. The resulting chromophore, p-nitroaniline, can be detected spectrophotometrically (Zhou et al., Anal. Biochem. 280(1) :159-165, 2000).

### 2.3.Kinetic Studies

Kinetic studies are performed to establish the velocity rates of the chemical reactions between enzymes and substrates under a number of conditions. “Kinetic constants” comprise proportionality constants that express the rate or specific reaction rate of a specific enzyme using a specific substrate. “Km” is defined as the substrate concentration at which the velocity of the enzyme is half maximal. It is normally used to compare conditions and substrates for a specific enzyme. These parameters are obtained by using Lineaweaver-Burk plots (Lehninher A. L., “Principles of Biochemistry”, Worth Publishers, New York, NT, pp:217-225.)

### 2.4. Activity of MetAPs in vitro in the Presence of Different Metals

In particular, the present invention illustrates that Mn²⁺ is one of the best stimulators for both MetAP1 and MetAP2 enzyme activity in vitro. Eight commonly used divalent metal ions were tested for their effect on both MetAP1 and MetAP2 activities. Co²⁺ and Mn²⁺ stood out as significant stimulators for both MetAP1 and MetAP2 activities on either MAS (methionine-alanine-serine) or MGAQFSKT (MARCKS protein) as substrate. Specifically, Mn²⁺ fully stimulated hMetAP2 both in the presence and absence of physiological concentration of glutathione (see Example 5). Identification of Mn²⁺ as one of the best stimulators for MetAPs enzyme activity is of great importance because Co²⁺ is considered to be physiologically irrelevant (Walker and Bradshaw, Biochem. Biophys. Acta (Review) 1477:157-167, 1998).

The present invention also includes the use of human microvascular endothelial cells, HMVEC, to test inhibitors or potential blockers of MetAP activity in whole cells. It is believed that these cells contain endogenous manganese in the active site. The specificity of endothelial cells inhibition by fumagillin and its synthetic analog TNP-40 has been reported elsewhere (Sin et al., Proc. Natl. Acad. Sci. USA, 94:6099-6103, 1997; Wang et al., J. Cell. Biochem. 77:465-473, 2000). The inhibition of endothelial cell proliferation has been correlated with the anti-angiogenic effect of fumagillin, TNP-40 or AGM-1470, the latter being currently under clinical trials for the treatment of several types of cancer. In addition to being used to test cell proliferation and angiogenesis, HMVEC are also used to determine specific MetAP2 activity. When HMVEC are grown under proper conditions, radioactive methionine is added to the cells with or without the inhibitory compounds to test for MetAP2 enzymatic activity on newly synthesized proteins, i.e., removal of N-terminal initiator methionine. The unprocessed initiator methionine reflects the inhibition of intracellular MetAP2 enzyme activity by MetAP2 inhibitors.

### 2.5. Angiogenesis in vitro

The invention further includes HMVEC cells sprout and tube formation in three-dimensional fibrin matrix, a useful angiogenesis model in vitro to test compounds that potentially inhibit MetAP and angiogenesis. Cells attached to a solid phase (for example microcarrier beads) embedded in a fibrin gel, grow and form tubule structures in the presence of appropriate angiogenesis stimulators. (Details are presented in Example 10). Using this model, inhibition of sprout and tube formation by a test compound indicates that the compound is a potential anti-angiogenic agent.

### 2.6. In vivo Models to Test MetAP Inhibitors

The present invention also includes in vivo models for angiogenesis and tumor growth performed in the presence of MetAP2 inhibitors to determine the correlation between the in vitro inhibitory activity of said compounds and their effects in tissues or whole animals. For example, a mouse cornea angiogenesis model is described therein in order to determine the effects of MetAP2 inhibitors on the cornea neo-vascularization in the presence of angiogenesis stimulators. (Details are presented in Example 11). Additionally, an athymic nude mice model is described therein to test tumor growth inhibition (See Example 12). In both models, administration of a compound that inhibits MetAP2, which retards angiogenesis or tumor formation, is considered to be a potential anti-angiogenic and anti-tumor compound through MetAP2 inhibition. The following examples are offered for illustrative purposes only and are not intended to limit the scope of the present invention in any way.

All U.S. patents and publications referred to herein are hereby incorporated in their entirety by reference.

### EXAMPLE 1

### Recombinant Human MetAP1 and MetAP2

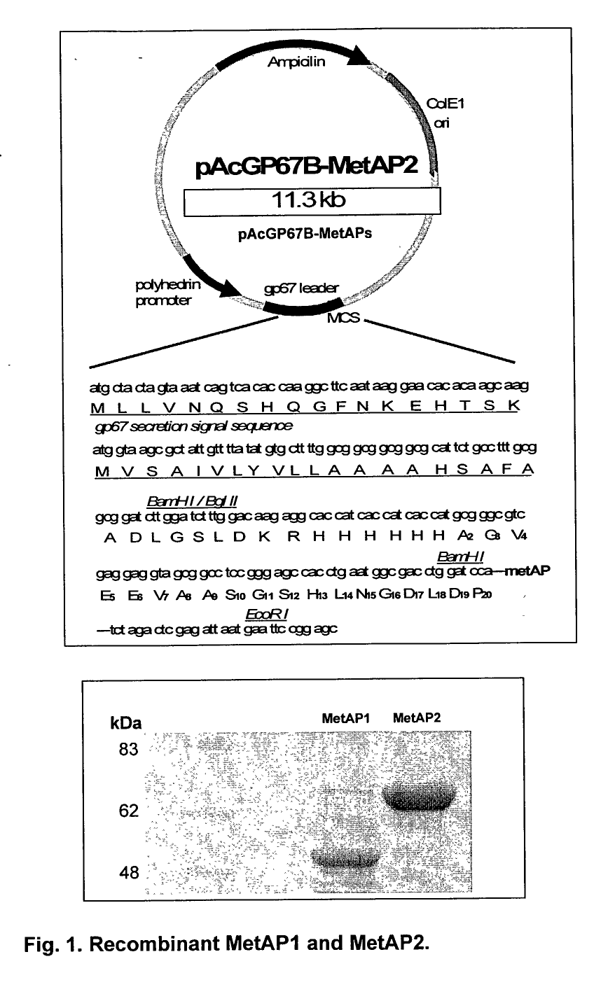

Both MetAP1 and MetAP2 enzymes with a N-terminal histidine tag were efficiently expressed and secreted by baculovirus infected Sf9 cells. MetAP2 cDNA was amplified from total RNA of human neonatal dermal microvascular endothelial cells (Clonetics, San Diego, Calif.) by RT-PCR with the following 2 oligonucleotide primers: 5′ - - - ATT AAT AGA TCT TTG GAC AAG AGG CAC CAT CAC CAT CAC CAT GCG GGC GTG GAG GAG GTA GCG GCC T - - - 3′ (SEQ ID NO:1); 5′ - - - ATT AAT CTC GAG TCT AGA CGG TCC GTT AAT AGT CAT CTC CTC TGC TGA CAA CT - - - 3′ (SEQ ID NO:2). Access RT-PCR kit (Promega, Madison, Wis.), 0.5 μg RNA per 50 μl and 1 μM primers were used in the one tube RT-PCR reaction according to manufacturer's instruction. The amplified DNA product was cloned into pCR-Blunt vector (Invitrogen, San Diego, Calif.), and its (pCR-Blunt-MetAP2) sequence was confirmed by DNA sequencing. MetAP2 cDNA was cut from pCR-Blunt-MetAP2 with Bgl II and EcoR I, and ligated to a baculovirus transfer vector pAcGP67B (Pharmingen, San Diego, Calif.) cut with BamH I (generating compatible ends to that of Bgl II) and EcoR I. The final expression vector pAcGP67B-MetAP2 is shown in FIG. 1.

Human MetAP1 cDNA sequence was reported in the literature as mRNA for KIAA0094. KIAA0094 is one of the forty new genes deduced by analysis of cDNA clones from human cell line KG-1 (Nagase et al, DNA Res. 2:37-43, 1995), which has complete 3′-end sequence of potential MetAP1 but not the defined 5′-end starting codon.

To clone full length MetAP1 cDNA, human fetal liver marathon-Ready™ cDNA library (Clontech, Palo Alto, Calif.) was used to carryout 5′-RACE with MetAP1 primer, 5′ - - - CGT TAA AAT TGA GAC ATG AAG TGA GGC CGT - - - 3′ (SEQ ID NO: 3), which is complementary to the 3′ end of MetAPl coding sequence. The PCR products were cloned into pT-Adv cloning vector (Clontech, Palo Alto, Calif.) and sequenced. The clone with the most extended 5′-end sequence had 40 bp additional sequences compared to the KIAA0094 sequence, and no new ATG codon upstream of the ATG codon (position 26) in KIAA0094 sequence was found, indicating KIAA0094 may already have the full length MetAP1 coding sequence. The MetAP1 coding sequence was then further PCR amplified with the following 2 primers: 5′ - - - ATT AAT GGA TCC A GCG GCC GTG GAG ACG CGG GTG T - - - 3′ (SEQ ID NO: 4) and 5′ - - - ATT AAT CTC GAG GAA TTC TTA AAA TTG AGA CAT GAA GTG AGG CCG T - - - 3′ (SEQ ID NO: 5). The resulting sequence was cut with BamH I and EcoR I and cloned to the baculovirus transfer vector pAcGP67B as shown in FIG. 1. Both MetAP2 and MetAP1 recombinant transfer vectors were transfected with BaculoGold® (Pharmingen, San Diego, Calif.) into insect SF9 cells, and recombinant viruses were obtained. Recombinant MetAP2 and MetAP1 were expressed and secreted into the culture medium of SF9 cells infected with the recombinant viruses.

### EXAMPLE 2

### Purification of Active MetAP1 and MetAP2

The serum-free culture media with expressed MetAP1 or MetAP2 resulting from Example 1, was diluted with an equal volume of cold water and loaded into a hydroxyapatite column equilibrated with 10 mM potassium phosphate buffer, pH 6.7, and MetAP2 or MetAP1 was eluted with a gradient of potassium phosphate buffer (10 mM to 400 mM). The fractions containing active MetAP2 or MetAP1 were pooled and diluted 5-fold with cold water and loaded into a cation exchange S20 column (BioRad, Hercules, Calif.) equilibrated with 10 mM Hepes buffer pH 7.4, 10 mM NaCl. MetAP2 or MetAP1 was eluted with a salt gradient (10 mM to 500 mM NaCl), and finally purified on a Sephacryl S-100 gel filtration column (Pharmacia, Piscataway, N.J.) with 10 mM Hepes buffer, pH 7.4, 150 mM NaCl. The purified MetAP1 (˜50 kDa) and MetAP2 (˜68kDa) were analyzed with SDS-PAGE as shown in FIG. 1. MetAP1 had an expected electrophoretic mobility for a 47-kDa protein, while MetAP2 had mobility of 67 kDa instead of theoretical 54 kDa (Gupta et al, In: Translational Regulation of Gene Expression (Ilan, J., Ed.) Vol. 2, pp 405-431, 1993). The unusual electrophoretic behavior of MetAP2 was documented in the literature when it was identified as p67 probably due to its elongated tracts of acidic and basic residues (Arfin et al., Proc. Nat'l Acad. Sci USA 16: 9261-4, 1995). Both recombinant MetAP1 and MetAP2 showed enzymatic activity in cleaving a tripeptide MAS substrate after purification on the nickel column. MetAP1 and MetAP2 are known to be metalloenzymes (Arfin et al., above; Li and Chang, Proc. Nat'l Acad. Sci USA 26:12357-61, 1996), and the presence of nickel ion may affect the activity of these enzymes.

In the present invention, a scheme was developed to purify MetAP1 and MetAP2 without any metal in the metal active site. Recombinant MetAP1 and MetAP2 expressed and purified without added exogenous metal ions in the culture medium or purification buffers possessed some baseline methionine aminopeptidase activity as seen with a coupled enzyme assay as described in the methods. MetAP1 had higher baseline activity than MetAP2. To minimize baseline activity of MetAP1 and MetAP2, both were treated with 5 mM EDTA followed by extensive dialysis to further ensure the absence of metal ions. These enzyme preparations, still possessing some baseline activity, were used for all of the following studies.

### EXAMPLE 3

### Activity Assays for MetAP1 and MetAP2

### 3.1.a. Chromogenic Assays

A coupled-enzyme chromogenic assay was used to measure methionine aminopeptidase activity by monitoring the production of free methionine cleaved from specific peptide substrates containing methionine. The peptide substrates for MetAPs include: the trimeric peptide MAS (methionine-alanine-serine) (Bachem, King of Prussia, Pa.) and other octameric peptide substrates based on the N-terminal sequence of human myristoylated proteins which were synthesized and HPLC purified by Research Genetics (Huntsville, Ala.) comprising methionine-glycine-alanine-glutamine-phenylalanine-serine-lysine-threonine (MARCKS proteins), methionine-glycine-asparagine-alanine₄-lysine (PKC-α), methionine-glycine-serine₂-lysine-serine-lysine-proline (Src^{p60}), methionine-glycine-asparagine-leusine-lysine-serine-valine-alanine (eNOS) and methionine-glycine-lysine-valine-lysine-valine-glycine-valine (GAPDH). Other peptides can be used including dimeric, tetrameric, pentameric, hexameric, heptameric, nonameric, decameric, and undecameric peptides. The trimeric MAS is commonly used in the literature, and the octamer methionine-glycine-alanine-glutamine-phenylalanine-serine-lysine-threonine is the natural N-terminal sequence of human MARCKS protein, a myristoylated alanine rich protein kinase C substrate (Resh M D, Biochem. Biophys.Acta (Review) 145(1):1-16, 1999). Free methionine was oxidized with L-amino acid oxidase (AAO, Sigma Catalog No. A-9378) and horseradish peroxidase (HRP, Sigma Catalog No. P-8451). Oxidation by AAO of free methionine generates hydrogen peroxide (H2O2) which then reacts with HRP oxidizing 0-dianisidine (Sigma Catalog No. D-1954) or 10-acetyl-3,7-dihydrophenoxazine (Amplex Red, Molecular Probes). Assays were performed in 96-well microtiter plates. Enzyme preparations were diluted in assay buffer (50 mM HEPES, pH 7.4, 100 mM NaCl), and 10 μl of the enzymes were introduced into each well. A mixture (90 μl) of 0.1 mg/ml L-amino acid oxidase, 0.1 mg/ml of horseradish peroxidase, 0.1 mg/ml ortho-dianisidine, and 0.5 mM peptide substrate was added to each cell. The reactions were carried out at room temperature, and the absorbance at 450 nanometer (A450) was measured every 20 seconds over a period of twenty minutes using an automatic plate reader (Molecular Devices, Calif., USA). The rate in mOD/min, calculated for each well, was used to represent MetAP1 or MetAP2 activity. Under this assay condition, the methionine cleaved from peptide substrate by MetAP1 or MetAP2 was instantaneously oxidized to generate a final A450 readout. It was determined that 1 μM methionine generated a 2.25 mOD increase in the A450 readout. These results allow for the correlation between the signal generated by the oxidation of cleaved free methionine and the activity of the MetAP1 and MetAP2.

### 3.1.b. Kinetic Studies with Different Peptides

MetAP2 using Mn²⁺ as cofactor was studied to obtain kinetic constants on several peptide substrates. Most of these peptides have the natural N-terminal sequences of myristoylated proteins. (Data is shown in Table 1). Enzyme kinetics was studied using the chromogenic assay to determine enzymatic activity as described in Example 3. Fourteen peptide substrates were used including MAS and several octameric peptides. These peptides were tested at a final concentration of 20, 40, 80, 160, 320, and 640 μM in the assay buffer (50 mM HEPES, pH 7.4, 100 mM NaCl). MetAP2 (final concentration of 30˜320 nM) was used in the presence of 100 μM of manganese chloride (Mn²⁺). The initial rate of methionine cleavage was determined by measuring the A450 change as described above. Kinetic constants were calculated using Linewaver-Burk plots. Removal of initiator methionine by MetAPs during protein translation occurs when the nascent peptide chain extends to 20-40 amino acid residues (Arfin et al., 1995). These data provide evidence that MetAp2 may use peptides of several lengths.

### 3.2. Radioactive Methionine Assays

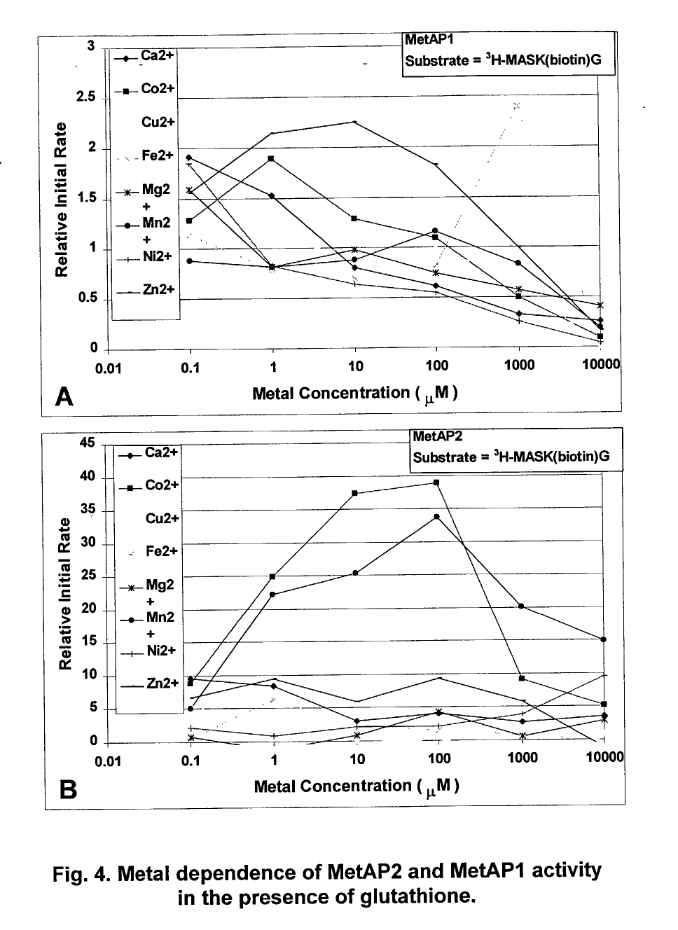

Another assay for methionine aminopeptidase activity involved the use of a peptide substrate containing ³H-methionine. ³H-Methionine-alanine-serine-lysine(biotin)-glycine-amide (³H-MASK(biotin)G) was synthesized in the lab on an Applied Biosystems “Synergy” peptide synthesizer using standard FMOC chemistry. Fmoc-L-Lys(biotinyl) —OH was purchased from Bachem (King of Prussia, Pa.). Fmoc-L-[methyl-³H]Methionine was synthesized by reacting Fmoc-Cl with L-[methyl-³H]Methionine (Amersham, Piscataway, N.J.) in a 1:1 Dioxane/10% Na₂CO₃(aq) mixed solvent. The peptides were deprotected and cleaved from the resin using a trifluoroacetic acid solution containing water, thioanisole, and ethanedithiol (900 μl:25 μl:50 μl:25 μl) as scavengers. The peptides were purified by HPLC using a Waters C18 Symmetry column (7.8×300 mm, 7 μm; Milford, Mass.). A gradient of acetonitrile/water (0.1% trifluoroacetic acid) was used from 3% to 15% acetonitrile in 20 minutes and a flow rate of 2 ml/min. Radioactive ³H-MASK(biotin)G peptide had specific activity of 13,000 cpm/nmol peptide; cleaved ³H-methionine was measured by scintillation counting after removal of the original peptide substrate with Streptavidin-agarose (Pierce, Rockford, Ill.). The resulting amount of cleaved radioactive methionine is directly proportional to the catalytic activity of the MetAP. Maximal stimulation of the enzyme results in increased release of free radioactive methionine (FIG. 4). (See U.S. Pat. No. 6,156,495 for a discussion of labels and uses thereof). Conversely, anything impairing the catalytic activity of MetAP (e.g., inhibitory agents, wrong metal cofactor) will result in a decreased amount of free radioactive methionine.

### EXAMPLE 4

### Effect of Metals on MetAP1 and MetAP2 Activities

Both MetAP1 and MetAP2 need the presence of a metal in the active site for optimal activity, and have previously been classified as Co²⁺, metalloproteases (Arfin et al, 1995; Li and Chang, 1996). However, the true identity of physiological relevant metal ions has been unclear and of controversy until now. The chromogenic assay with either the MAS peptide or MGAQFSKT peptide described in Example 3.1, was used to study the effect of metals on MetAP1 and MetAP2 activities. Calcium chloride (Ca²⁺), cobalt chloride (Co²⁺), cupric chloride (Cu²⁺), ferrous chloride (Fe²⁺), magnesium chloride (Mg²⁺), manganese chloride (Mn²⁺), nickel chloride (Ni²⁺), or zinc chloride (Zn²⁺) was included in each assay at final concentrations of 0, 0.1, 1, 10, 100, 1000, and 10,000 μM in the assay buffer (50 mM HEPES, pH 7.4, 100 mM NaCl).

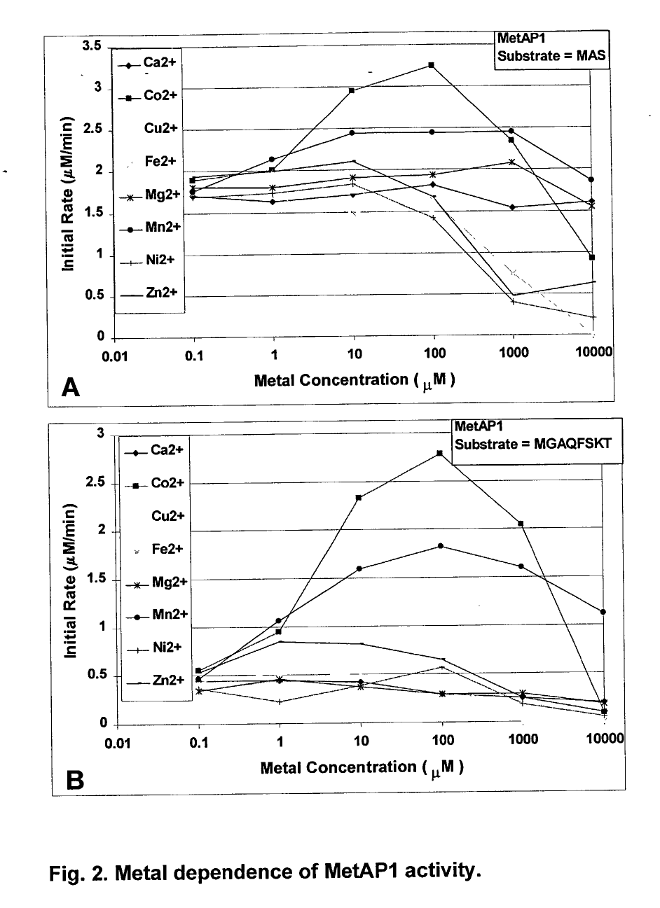

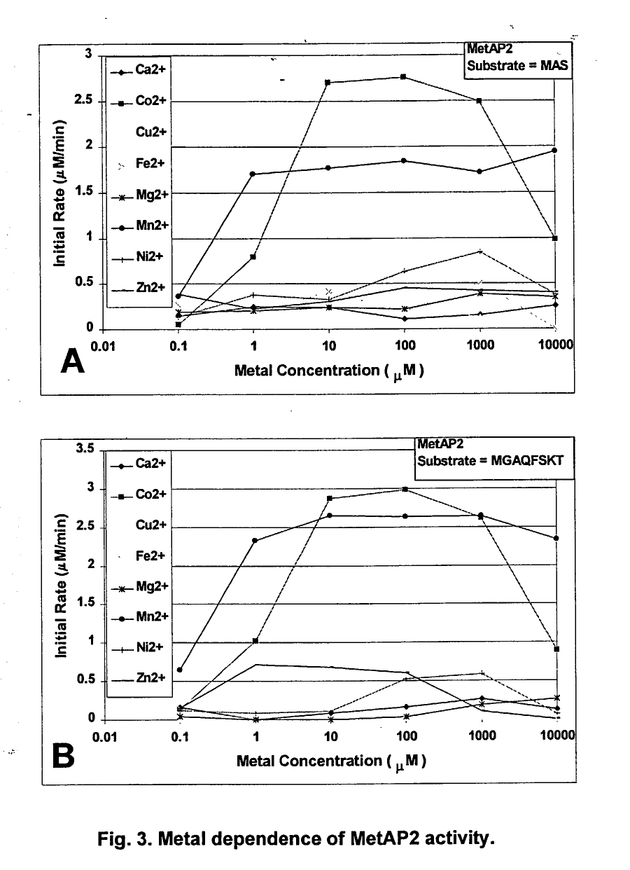

The results of the coupled enzyme chromogenic assays using the two peptide substrates with different length are shown in FIGS. 2 and 3. FIG. 2 shows that MetAP1 had 14-fold higher baseline activity (1.68 μM/min turnover at 120 nM MetAP1) on the short MAS peptide than on the longer peptide MGAQFSKT (0.51 μM/min turnover at 500 nM MetAP1). MetAP1 had a high baseline activity with MAS substrate, and stimulation of its activity by Co²⁺ and Mn²⁺ was about 2-fold (FIG. 2A). When MGAQFSKT was used as the substrate, MetAP1 was stimulated 5-fold by Co²⁺ and 3-fold by Mn²⁺(FIG. 2B). FIG. 3 shows that the baseline activity of MetAP2 was minimal and did not show a significant difference on the two substrates (0.19 μM/min turnover of MAS at 60 nM MetAP2 compared to 0.12 μM/min turnover of MGAQFSKT at 30 nM MetAP2). Stimulation of MetAP2 activity was greatly increased and a lower metal concentration was required for maximum effect. Both Co²⁺ and Mn²⁺, stimulated MetPA2 activity on MAS and MGAQFSKT substrates 10 to 20-fold (FIGS. 3A and 3B). Co²⁺ showed maximum effect at 10 μM while Mn²⁺ reached its maximum effect at 1 μM. At higher concentrations, all the metal ions, except Mn²⁺, showed inhibitory activity on the enzymes.

### EXAMPLE 5

### Metal Dependence of MetAP1 and MetAP2 in the Presence of Glutathione

To study the effect of metal ions in the presence of physiologically relevant reduced glutathione (GSH) (Walker and Bradshaw, 1998), the assay measuring radioactive methionine release from a peptide described in Example 3.2 was used because GSH affected the coupled enzyme chromogenic assay. In the presence of 5 mM glutathione, both Co²⁺ and Mn²⁺ were able to stimulate MetAP2 activity by 30˜40-fold (FIG. 4A). At low concentrations (0.1˜1 μM), Zn²⁺ and Ca²⁺ also enhanced MetAP2 activity 5˜10-fold. The effect of metal ions on MetAP1 activity in the presence of 5 mM glutathione (FIG. 4B) was different from that without glutathione (FIG. 2). In particular, Zn²⁺ showed the best activity while Co²⁺ and Fe²⁺ were also stimulatory.

Mn²⁺ fully stimulated human MetAP2 activity at a concentration as low as 1 μM and had a broader window of activating concentrations both in the presence and absence of physiological concentrations of GSH. E. coli MetAP1 (kindly provided by Dr. B. Mattews, Institute of Molecular Biology, University of Oregon) behaved similarly to human MetAP1, capable of using Mn²⁺ and Co²⁺ as cofactors (data not shown) in the absence of glutathione.

### EXAMPLE 6

### Cellular MetAP Activity and Effect of Inhibitors

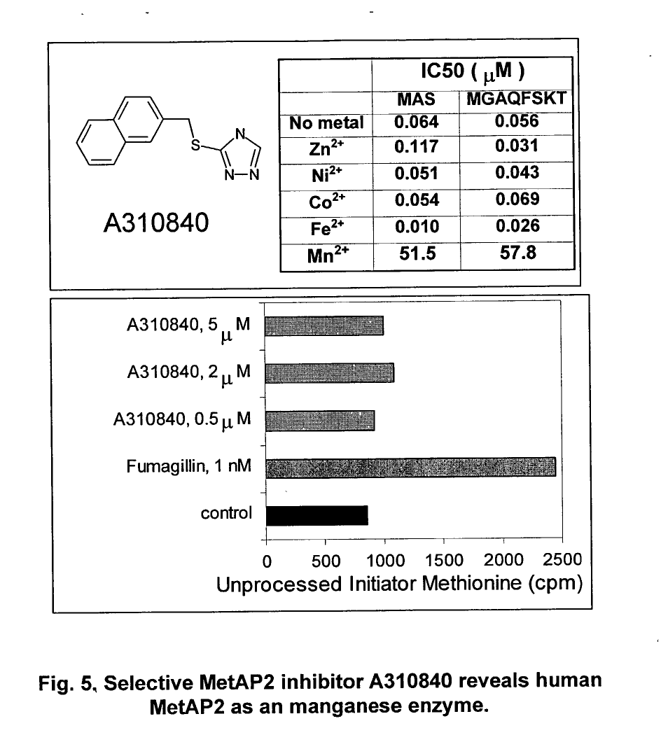

Human microvascular endothelial cells (HMVEC) (Clonetics, San Diego, Calif.) were grown in EGM2 recommended medium (Wang et al., J. Cell. Biochem. 77: 465-473, 2000). Cells in T75 flasks at approximately 70% confluence were changed to 15 ml EGLM (labeling medium, Clonetics) without added methionine. Fumagillin (Sigma Catalog No. F-6771) or A310840 (Abbott Laboratories, Abbott Park, Ill.) was added to the culture to final concentrations of 1 nM (fumagillin) or 0.5, 2 and 5 μM (A310840). The cells and inhibitors were incubated for four hours before adding 0.5 mCi/flask of ³⁵S-methionine (Amersham, Piscataway, N.J.). The cells were allowed further incubation for two hours, washed and lysed in 1 ml M-per (Pierce, Rockford, Ill.) buffer. The lysate samples having the same amount of radioactivity were loaded to a 0.5 ml column of Reactive Blue 72-agarose (Sigma, St. Louis, Mo.) equilibrated with 20 mM Tris/HCl, pH 7.5, 150 mM NaCl. The columns were washed with 30 ml equilibrium buffer, and then incubated with 3 ml of 100 nM MetAP2 in equilibration/washing buffer for 20 minutes at room temperature. The MetAP2 cleaved radioactivity (unprocessed initiator methionine) was then counted. Selective inhibition of MetAP2 in HMVEC by fumagillin resulted in an increased unprocessed initiator methionine from cellular proteins (see FIG. 5).

### EXAMPLE 7

### Synthesis of a Triazole MetAP2 Inhibitor

### A310840 (3-((2-naphthylmethyl)sulfanyl)-4H-1,2,4-triazole)

2-(bromomethyl)naphthalene (0.38 g, 1.7 mmol) was added to a suspension of 3-mercapto-1,2,4-triazole (0.18 g, 1.8 mmol) and cesium carbonate (0.72 g, 2.2 mmol) in 5 mL of N,N-dimethylformamide. The mixture was heated at 40° C. for 16 hours. The volume was reduced by rotary evaporation, and the remaining mixture was shaken with water and methylene chloride, and then filtered. The layers of the filtrate were separated, and the organic phase was dried over magnesium sulfate. Filtration and solvent removal gave a white solid (0.143 g). MS (DCI/NH3) m/e 242 (M+H)⁺, 259 (M+NH4)⁺; ¹H NMR (300 MHz, DMSO-d₆) δ14.08 (s, 1H) , 8.57 (bds, 1H) , 7.88 (m, 4H), 7.49 (m, 3H), 4.51 (s, 2H); Anal. calcd for C10H20N4S: C, 64.70; H, 4.59; N, 17.41. Found: C, 64.93; H, 4.58; N, 17.25. The resulting compound is a selective inhibitor of MetAP2 with various metal cofactors, and it was used to establish the specific metal cofactor for MetAP2.

### EXAMPLE 8

### Use of a Selective Inhibitor A310840 to Show MetAP2 as a Manganese Enzyme

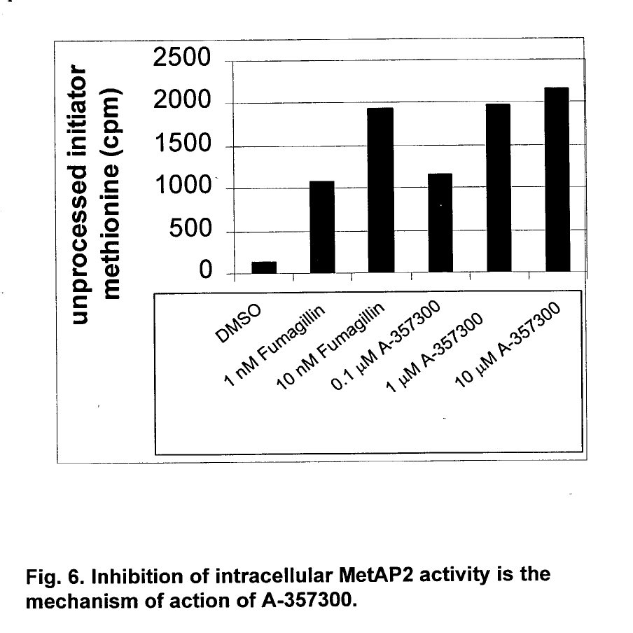

A310840 (Abbott Laboratories, Abbott Park, Ill.) is a triazole MetAP2 inhibitor with potent inhibitory activity on the baseline activity (without added metals) of MetAP2 (IC50=60 nM) and on MetAP2 with Co²⁺ (IC50=61 nM) or many other metal ions including Zn²⁺, Fe²⁺, and Ni²⁺ (IC50=10˜117 nM). A310840, however, is 1000 fold less active as an inhibitor of MetAP2 when the enzyme is using Mn²⁺ as the metal cofactor (IC50=50 μM) (FIG. 5). This selective inhibition of MetAP2-Co²⁺ and other metal forms by A310840 was not affected by the peptide substrates used. A310840 was able to penetrate and accumulate into HMVEC cells, but it did not inhibit the cell proliferation (data not shown). Analysis of cellular proteins including GAPDH for N-terminal methionine status revealed that processing of protein initiator methionine was not blocked by the treatment of HMVEC with A310840 up to 10 μM, while treatment of HMVEC with 1 nM fumagillin, a covalent MetAP2 inhibitor that inhibited MetAP2 with all metal ions, resulted in inhibition of cell proliferation and accumulation of unprocessed N-terminal methionine of cellular proteins. Like fumagillin, A357300 (another MetAP2 inhibitor, see Example 9) showed inhibition of MetAP2-Mn²⁺, cellular protein initiator methionine processing and cell proliferation (See FIG. 6). These data indicated that cellular MetAP2 was not functioning as a Co²⁺ enzyme but as a Mn²⁺ enzyme, indicating that cellular MetAP2 was using manganese as a cofactor. Direct evidence on the MetAP2 metal cofactor identity is still lacking. Purification of MetAP2 from a natural source without extra metal contamination and chelating effect during the process should allow true metal identification. However, based on the characterization of the enzyme in vitro and the application of A310840 to evaluate cellular MetAP2 enzyme function, it appears that the cellular metal ion for human MetAP2 is manganese.

### EXAMPLE 9

### A-357300 Inhibition of Endothelial Cell Proliferation

A-357300 is a newly discovered reversible inhibitor of MetAP2 that selectively inhibits MetAP2. It selectively inhibited MetAP2 catalytic activity with an IC₅₀ of 0.117 μM when the enzyme was using Mn²⁺ and with an IC₅₀ of 0.078 μM when the enzyme was using Co²⁺. A-357300 inhibited MetAP1 with an IC₅₀ of 56.7 μM and did not inhibit other aminopeptidases such as leucine aminopeptidase at a concentration below 100 μM (data not shown). The cellular activity of A-357300 was tested in HMVEC grown in complete EGM2 medium enriched with a combination of growth factors (vascular endothelial growth factor (VEGF), basic fibroblast growth factor (bFGF), epidermal growth factor (EGF), and insulin-like growth factor (IGF)) and fetal bovine serum. In a 3-day proliferation assay, A-357300 showed a concentration dependent inhibition of HMVEC proliferation with an IC₅₀of 0.1 μM. To determine that the cellular effect of A-357300 was through inhibition of cellular MetAp2, HMVEC were incubated with the MetAP2 inhibitors fumagillin and A-357300 for 4 hours before ³⁵S-methionine was added to the culture. After another 2-hour incubation, cellular proteins were collected and applied to a reactive dye-agarose column as described in Example 6. MetAP2 specific substrates (GAPDH and other cellular proteins with a N-terminal cleavable initiator methionine) were captured on the agarose column. Increases in this exogenous MetAP2 cleavable initiator methionine (i.e., the unprocessed initiator methionine) reflected the inhibition of cellular MetAP2 enzyme activity by inhibitors. A-357300, like fumagillin, was able to block cellular MetAP2 enzyme activity at concentrations that were effective in inhibiting cell proliferation. This indicates that the mechanism of action of these compounds in inhibiting cell proliferation is through inhibition of MetAP2 enzymatic activity.

Table 2 summarizes the selectivity of MetAP2 inhibitors in inhibiting the proliferation of endothelial cells and certain tumor cells. Proliferation of endothelial cells of various origins, normal human primary cells pf non-EC type, and tumor cell lines were studied with MetAP2 inhibitors A-357300 and fumagillin. IC₅₀ of proliferation inhibition of these cells are listed in the table. MetAP2 inhibitors selectively inhibited endothelial cells, but not normal primary cells of other cell types. Most tumor cells were sensitive to MetAP2 while some were resistant.

### EXAMPLE 10

### A-357300 Inhibition of Angiogenesis in vitro

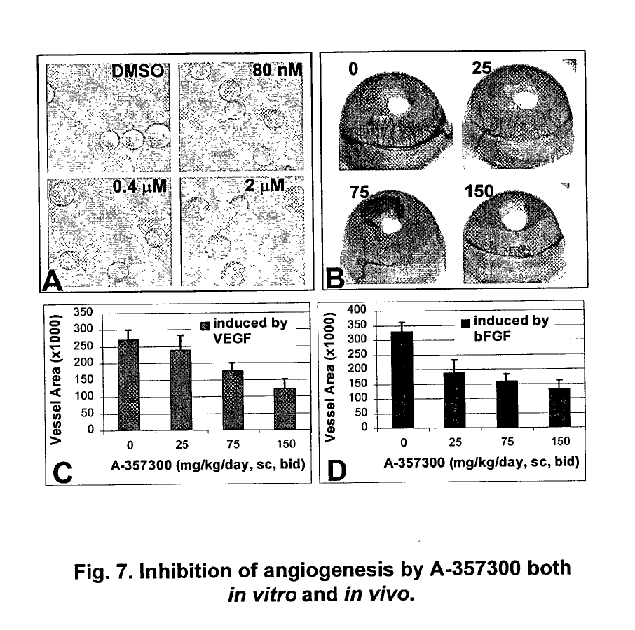

To determine the inhibitory effect of A-357300 on angiogenesis, the sprout and tube formation of endothelial cells angiogenesis model was used. HMVEC cells attached to microcarrier beads embedded in a fibrin gel, grow, migrate, sprout and form tubule structures in the presence of angiogenesis stimulators like VEGF and bFGF. HMVEC cells mixed with gelatin coated Cytodex Microcarrier Beads (Sigma, Piscataway, N.J.) at 1×10⁶ cells/ml and 30% (v/v) beads in EGM2 media (containing growth factors and 5% bovine serum albumin) were incubated at 37° C. and 5% CO2 for 4 hours. After incubation cells/beads are normally confluent and were resuspended in fresh media at 1%(v/v) and mixed with an equal volume of 6 mg/ml human fibrinogen (Sigma) in EBM2 basic medium. Human thrombin (Sigma) was added to a final concentration of 0.05 U/ml, and the mixture was dispersed to a 24-well plate (1 ml/well). After 2-3 days, in the presence of angiogenesis stimulators VEGF and bFGF, sprout and tube formation was checked under a phase contrast microscope. A-357300 completely blocks the formation of the structures in HMVEC at concentrations of 0.4 μM and 2 μM (FIG. 7A).

### EXAMPLE 11

### A-357300 Inhibition of Angiogenesis in vivo

The effect of A-357300 on in vivo angiogenesis was determined in the mouse cornea angiogenesis model. Subcutaneous injections of A-357300 at 25, 75 and 150 mg/kg/day twice daily inhibited growth factor cornea neovascularization in a dose-dependent manner against VEGF and bFGF. Plasma drug concentrations measured at the 6-hour time point after the terminal dose were 0.24, 0.38 and 2.2 μM, respectively, for each of the groups mentioned above, and correlated to the efficacy of each dose group. Cornea angiogenesis induced by pro-angiogenic agent bFGF was reduced by half in the presence of 25 mg/kg/day A-357300 (See FIG. 7D). Higher concentrations resulted in higher inhibition of corneal neovascularization and vessel area (see FIGS. 7B, 7C, 7D). These results highlight the finding that inhibition of angiogenesis in vivo results from inhibition of MetAP2.

### EXAMPLE 12

### A-357300 Inhibition of Tumor Growth in vivo

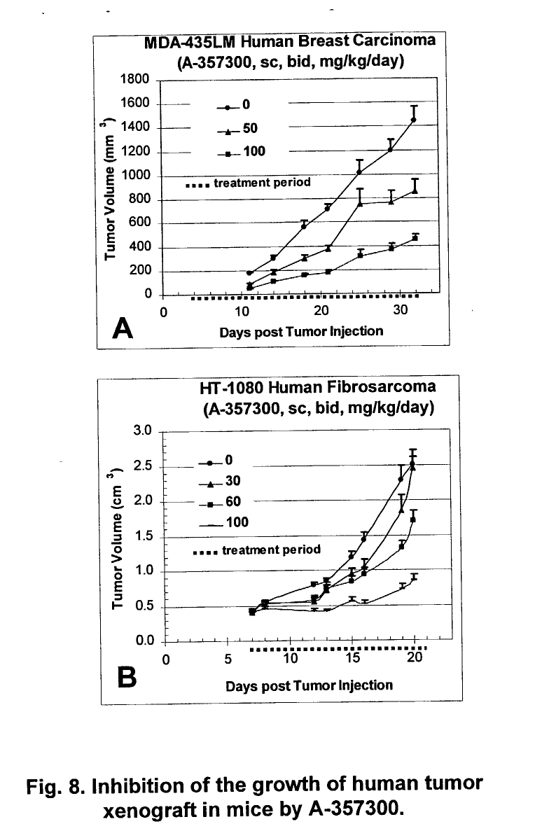

The in vivo efficacy of A-357300 as an antitumor agent was measured in athymic nude mice carrying certain tumor cells in the subcutaneous flank. Athymic nude mice carrying CHP-134 human neuroblastoma xenograft were previously used to evaluate TNP-470 anti-tumor activity (Shusterman et al., Clin. Cancer Res. 7(4) :977-84, 2001). Experiments using MDA-435 LM human breast carcinoma and HT-1080 human fibrosarcoma xenografts were performed to determine A-357300 anti-tumor activity in vivo. Cells were grown in RPMI 1640 (Life Technologies, Inc., Rockville, Md.) containing 10% fetal bovine serum and 1% L-glutamine, penicillin, streptomycin, and oxalacetic acid-pyruvate-insulin (OPI 100×; Gibco, Grand Island, N.Y.). Cells were passaged when they reached near 100% confluence. Six-week old athymic (nu/nu; NCI, Frederick, Md.) mice were used for xenografting. Cell suspensions were injected with a 26-gauge needle into the right flank of the mice, raising a wheal. Tumor growth was observed within 14 days following inoculation in 90% of animals. Treatment was initiated when the tumor reached 0.2 cm³ in size. MetAP2 inhibitors were administered subcutaneously twice daily, and an equivalent volume of HPMC was given to control mice. Treatment was continued for 30 days or until tumor volume exceeded 3.0 cm³. Animals were sacrificed at this time and necropsied. Tumor growth rate was markedly inhibited in mice with MDA-435LM human breast carcinoma receiving A-357300 at 100 mg/kg/day sc dose starting at day 12 (FIG. 8A) These results indicate that selective inhibition of MetAP2 by A-357300 (as shown in vitro in Example 11) results in reduction of tumor volume and growth. Similarly, tumor growth rate was markedly inhibited by day 12 in mice with HT-1080 human fibrosarcoma when 100 mg/Kg/day was administered (FIG. 8B).

TABLE 1
Kinetic constants for MetAP2 using different length peptides

| | Km | kcat | |
|---|---|---|---|
| | μM | 1/min | kcat/Km |
| MAS | 211.5 | 152.3 | 0.72 |
| MGK | 757.6 | 199.4 | 0.3 |
| MGKV | 623.7 | 402.1 | 0.6 |
| MGKVK | 188.1 | 626.6 | 3.3 |
| MGKVKV | 149.3 | 511.6 | 3.4 |
| MGKVKVG (GADPH) | 215.4 | 573.8 | 2.7 |
| MGKVKVGVN | 205.0 | 611.4 | 3.0 |
| MGKVKVGVNG | 163.6 | 610.4 | 3.7 |
| MGKVKVGVNGF | 131.3 | 565.5 | 4.3 |
| MGAQFSKT (MARCKS protein) | 224.4 | 292.2 | 1.3 |
| MGNAAAAK (PKC a) | 259.9 | 226.9 | 0.9 |
| MGSSKSKP (Src p60) | 233.1 | 236.2 | 1.0 |
| MGNLKSVA (eNOS) | 208.3 | 292.2 | 1.4 |

TABLE 2
MetAP2 inhibitors selectively inhibit the proliferation
of EC and certain tumor cells.

IC50s (_{μ}M)

| Cell | Type | A-357300 | Fumagillin |
|---|---|---|---|
| EC | HMVEC/HUVEC | 0.1 | 0.001 |
| | Human CEC | 0.1 | 0.001 |
| | BEND3 (mouse) | 0.2 | 0.002 |
| | BAEC (bovine) | 0.1 | 0.001 |
| Normal | Mammary Epithelial cells | >100 | >1 |
| Human | Prostate Epithelial cells | >100 | >1 |
| Primary | Astrocytes | 53.6 | >1 |
| Cells | T-lymphocytes | 19.41 | 8.3 |
| | Fibroblasts | 2 | >1 |
| Tumor | PC3 | >100 | >1 |
| Cells | MCF7 | 100 | >1 |
| | MDA-MB | 24 | >1 |
| | MDA-435-LM | 2 | >1 |
| | MiaPaCa2 | 1 | >1 |
| | A549 | 0.2 | 0.002 |
| | DLD-1 | 0.2 | 0.002 |
| | HCT-15 | 0.2 | 0.003 |
| | NCI-H460 | 0.2 | 0.003 |
| | HT-1080 | 0.1 | 0.001 |
| | CHP-134 | 0.1 | 0.001 |

1) A method for assaying the activity of an aminopeptidase, comprising the steps of contacting said aminopeptidase with a substrate comprising methionine for a time and under conditions sufficient to allow said aminopeptidase to cleave said substrate in order to release said methionine, in the presence of the metal cofactor manganese, wherein the cleavage of said methionine generates a measurable signal, wherein said measurable signal indicates activity of said aminopeptidase.

2) The method of claim 1, wherein the aminopeptidase is a methionine aminopeptidase.

3) The method of claim 2, wherein the methionine aminopeptidase is selected from the group consisting of methionine aminopeptidase Type 2 and methionine aminopeptidase Type 1.

4) The method of claim 3, wherein the methionine aminopeptidase Type 2 is human methionine aminopeptidase Type 2.

5) The method of claim 1, wherein the substrate is an oligomeric peptide.

6) The method of claim 5, wherein the oligomeric peptide is selected from the group consisting of trimeric tetrameric, pentameric, hexameric, heptameric, octameric, nonameric, decameric, and undecameric peptides.

7) The method of claim 6, wherein the trimeric peptide comprises methionine-alanine-serine (MAS) and methionine-glycine-lysine (MGK).

8) The method of claim 6, wherein the octameric peptide is selected from the group consisting of methionine-glycine-alanine-glutamine-phenylalanine-serine-lysine-threonine (MARCKS proteins), methionine-glycine-asparagine-alanine₄-lysine (PKC-α), methionine-glycine-serine₂-lysine-serine-lysine-proline (Src^{p60}), methionine-glycine-asparagine-leusine-lysine-serine-valine-alanine (eNOS) and methionine-glycine-lysine-valine-lysine-valine-glycine-valine (GAPDH).

9) The method of claim 1, wherein the metal cofactor is manganese in divalent form.

10) The method of claim 1, wherein the measurable signal results from detection of free radioactive methionine released upon enzymatic activity of said aminopeptidase on a substrate comprising radioactive methionine.

11) The method of claim 10, wherein the radioactive methionine is selected from the group consisting of ³H-methionine, ³⁵S-methionine, and ¹⁴C-methionine.

12) The method of claim 1, wherein the measurable signal results from detection of color development resulting from free methionine released from a substrate upon activity of said aminopeptidase.

13) The method of claim 12, wherein said color development results from oxidation of said free methionine.

14) The method of claim 1, wherein a tetrapeptide comprising methionine is cleaved by the aminopetidase and the resulting methionine-free tripeptide and free methionine are separated by high pressure liquid chromatography (HPLC).

15) The method of claim 14, wherein the measurable signal results from the generated methionine-free tripeptide.

16) The method of claim 1, wherein the substrate is a peptide selected from the group consisting of methionine-p-nitroanilide (Met-pNA) and L-methionine 7-amido-4-methylcoumarin (Met-AMC).

17) The method of claim 16, wherein methionine is cleaved by the aminopeptidase and the measurable signal results from detection of color development resulting from methionine-free p-nitroanilide (pNA).

18) The method of claim 16, wherein methionine is cleaved by the aminopeptidase and the measurable signal results from detection of fluorescence resulting from methionine-free 7-amido-4-methylcoumarin (AMC).

19) A method for assaying the activity of methionine aminopeptidase, comprising the steps of:
(a) contacting said methionine aminopeptidase with a first substrate comprising methionine for a time and under conditions sufficient to allow said methionine aminopeptidase to cleave said first substrate in order to release said methionine, in the presence of the metal cofactor manganese, wherein cleavage of said methionine generates a second substrate,
(b) contacting said second substrate with a peptidase other than methionine aminopeptidase, wherein said peptidase other than methionine aminopeptidase is capable of generating a measurable signal,
wherein said measurable signal indicates activity of said methionine aminopeptidase.

20) The method of claim 19, wherein the first substrate in step (a) is a dipeptide comprising methionine.

21) The method of claim 20, wherein the dipeptide is Met-Pro-p-nitroanilide.

22) The method of claim 19, wherein the peptidase in step (b) is a proline aminopeptidase.

23) A method for identifying compounds that inhibit function of aminopeptidase comprising the steps of:
(a) contacting an aminopeptidase with a polypeptide comprising labeled methionine in the presence of divalent manganese as a metal cofactor, wherein said manganese is either exogenously added or is complexed to said aminopeptidase;
(b) allowing the (1) contacted aminopeptidase, (2) polypeptide comprising labeled methionine, and (3) divalent manganese to react for a time and under conditions sufficient for said aminopeptidase to cleave said labeled methionine from said polypeptide;
(c) measuring the amount of cleaved labeled methionine by detecting the amount of signal generated by said label;
(d) performing steps (a), (b) and (c) in the presence of a test compound, and measuring said resulting signal from step (c),
(e) comparing signals generated by steps (c) and (d), wherein a decreased signal in step (d) compared to said signal in step (c) generated in the presence of said test compound, indicates said test compound is an inhibitor of said metalloprotease when manganese is the metal cofactor.

24) The method of claim 23, wherein labeled methionine is labeled with a radioisotope.

25) The method of claim 24, wherein said radioisotope is selected from the group consisting of tritium ³[H]), ³⁵[S] and ¹⁴[C].

26) A method for identifying compounds that inhibit function of metalloprotease comprising the steps of:
(a) contacting a metalloprotease with a polypeptide comprising methionine in the presence of divalent manganese as a metal cofactor, wherein said manganese is either exogenously added or is complexed to said metalloprotease;
(b) allowing the (1) contacted metalloprotease, (2) polypeptide comprising methionine and, (3) divalent manganese to react for a time and under conditions sufficient for said metalloprotease to cleave said methionine from said polypeptide;
(c) adding a first enzyme to said cleaved methionine, wherein said first enzyme oxidizes said cleaved methionine thereby producing H₂O₂;
(d) measuring the amount of said cleaved methionine by determining the amount of H₂O₂ produced by said oxidation reaction;
(e) adding a second enzyme for which said H₂O₂ is a substrate, resulting in the production of an oxidizing agent that generates a measurable signal upon oxidation of a signal-generating agent;
(f) performing steps (a) through (e) in the presence of a test compound, and measuring said resulting signal from step (f);
(g) comparing the signals generated by steps (e) and (f), wherein a decreased signal generated in step (f) in the presence of said test compound as compared to said signal of step (e), indicates said test compound is an inhibitor of said metalloprotease when manganese is the metal cofactor.

27) The method of claim 26, wherein the oxidizing enzyme of step (c) is L-amino oxidase.

28) The method of claim 26, wherein the second enzyme in step (e) is horseradish peroxidase.

29) The method of claim 26, wherein the signal-generating agent of step (e) is selected from the group consisting of o-dianisidine and Amplex Red.

30) A method for identifying compounds that inhibit function of metalloprotease comprising the steps of:
(a) contacting a metalloprotease with a substrate comprising methionine in the presence of divalent manganese as a metal cofactor, wherein said manganese is either exogenously added or is complexed to said metalloprotease;
(b) allowing the (1) contacted metalloprotease, (2) substrate comprising methionine and, (3) divalent manganese to react for a time and under conditions sufficient for said metalloprotease to cleave said methionine from said substrate;
(c) measuring the amount of methionine free-substrate by detecting a measurable signal generated by said methionine-free substrate;
(d) performing steps (a), (b), and (c) in the presence of a test compound, and measuring said resulting signal as in step (c);
(e) comparing the signals generated by steps (c) and (d), wherein a decreased signal generated in step (d) in the presence of said test compound as compared to said signal of step (c), indicates said test compound is an inhibitor of said metalloprotease when manganese is the metal cofactor.

31) The method of claim 30, wherein the substrate comprising methionine is selected from the group comprising L-methionine p-nitroanilide and L-methionine 7-amido-4-methylcoumarin.

32) The method of claim 31 wherein the substrate comprising methionine is L-methionine p-nitroanilide and the measurable signal results from color development from the methionine free substrate p-nitroaniline.

33) The method of claim 31 wherein the substrate comprising methionine is L-methionine 7-amido-4-methylcoumarin and the measurable signal results from the fluorescent methionine free substrate 7-amido-4-methylcoumarin.

34) A method to identify compounds that inhibit function of metalloprotease comprising the steps of:
(a) contacting a metalloprotease with a tetrapeptide comprising methionine as the substrate, in the presence of divalent manganese as a metal cofactor, wherein said manganese is either exogenously added or is complexed to said metalloprotease;
(b) allowing the (1) contacted metalloprotease, (2) substrate comprising methionine and, (3) divalent manganese to react for a time and under conditions sufficient for said metalloprotease to cleave said methionine from said substrate;
(c) measuring the amount of methionine free-substrate after separation by HPLC by measuring the signal generated by said methionine-free substrate;
(d) performing steps (a), (b), and (c) in the presence of a test compound, and measuring said resulting signal as in step (c);
(e) comparing the signals generated by steps (c) and (d), wherein a decreased signal generated in step (d) in the presence of said test compound as compared to said signal of step (c), indicates said test compound is an inhibitor of said metalloprotease when manganese is the metal cofactor.

35) A method for determining intracellular MetAP2 inhibition by a compound, wherein said compound inhibits aminopeptidase activity in a test cell comprising endogenous manganese as a metal cofactor, comprising the steps of:
(a) contacting a test cell with labeled methionine for a time and under conditions sufficient to allow said test cell to incorporate said radioactive methionine into proteins produced by said test cell;
(b) isolating said produced proteins;
(c) contacting said produced proteins with exogenous aminopeptidase-manganese complex for a time and under conditions sufficient to cleave labeled N-terminal initiator methionine from said produced proteins;
(d) determining the amount of labeled methionine cleaved in step (c);
(e) repeating step (a) in the presence of a test compound, and then repeating steps (b) through (d);
(f) comparing the amount of cleaved radioactive methionine from steps (d) and (e), wherein an increase of free labeled methionine in step (e) as compared to step (d) indicates that said test compound has intracellular MetAP2 inhibitory activity.

36) The method of claim 35, wherein the test cell is selected from the group consisting of an endothelial cell (HMVEC), a tumor cell and a white blood cell.

37) A method for determining anti-angiogenic activity of a compound in vitro, wherein said compound inhibits aminopeptidase activity in an endothelial cell, comprising the steps of:
contacting an endothelial cell with a compound that inhibits methionine aminopeptidase activity and determining whether said compound inhibits endothelial cell proliferation, wherein lack of proliferation indicates said compound has anti-angiogenic activity.

38) The method of claim 37, wherein the endothelial cell is a Human Microvascular Endothelial cell (HMVEC).

39) A method for determining anti-tumor activity of a compound in vitro, wherein said compound inhibits aminopeptidase activity in a tumor cell, comprising the steps of:
contacting a tumor cell with a compound that inhibits methionine aminopeptidase activity and determining whether said compound inhibits tumor cell proliferation, wherein lack of proliferation indicates said compound has anti-tumor activity.

40) A method of inhibiting methionine aminopeptidase activity in a mammal in need of said inhibition, comprising administering to the mammal a therapeutically effective amount of a compound that inhibits methionine aminopeptidase activity.

41) A method of treating or preventing angiogenesis in a mammal in need of said treatment or prevention comprising administering to said mammal a therapeutically effective amount of a compound that inhibits methionine aminopeptidase activity.
